(19) United States
(12) Patent Application Publication
(10) Pub. No.: US 2025/0073039 A1
(43) Pub. Date: Mar. 6, 2025

(54) SACROILIAC FUSION IMPLANT

(71) Applicants: Steven Craig Meyer, Columbia, MO (US); Greg Logsdon, Columbia, MO (US); Brion Daffinson, Marietta, GA (US); Austin Howell, Lakewood, CO (US)

(72) Inventors: Steven Craig Meyer, Columbia, MO (US); Greg Logsdon, Columbia, MO (US); Brion Daffinson, Marietta, GA (US); Austin Howell, Lakewood, CO (US)

(21) Appl. No.: 18/460,074

(22) Filed: Sep. 1, 2023

(51) Int. Cl. A61F 2/30

(52) U.S. Cl. CPC A61F 2/30988; A61F 2002/30011; A61F 2002/30593; A61F 2002/30622; A61F 2002/3092; A61F 2002/30968; A61F 2002/3097; A61F 2002/30995; A61F 2002/4629; A61F 2002/4677

(57) ABSTRACT

A sacroiliac fusion implant device being unitarily formed in a single piece construction using an additive manufacturing process. The implant extends along a longitudinal axis. The implant has a central core, a first latticed volume and a second latticed volume. The central core has a proximal end, a distal end and a tube body disposed between the proximal end and the distal end. The central core has a lumen extending along the longitudinal axis from a proximal end opening to a distal end opening configured to pass over a guide wire or pin. The distal end is tapered extending to the distal end opening. The proximal end has external threads configured for instrument engagement. The tube body has three transverse elongated longitudinal slots or openings that extend across the lumen.

### FIELD OF THE INVENTION

The present invention relates to a novel sacroiliac fusion implant device.

### BACKGROUND OF THE INVENTION

The sacroiliac or SI joint is a very important joint in the lower back with one joint on each side of the pelvis basically connecting the base of the spine to the pelvis. The sacroiliac joint is a low-motion joint that connects the hip bones to either side of the sacrum. It is formed by the tailbone, sacrum, and waist bones, ilium, and functions as a shock absorber between the spine and legs. It is a generally C shaped joint with cartilage and an extensive complex of supporting ligaments.

One of the functions of the SI joint is a shock absorber. It transfers the forces from the upper body to the lower body. As such it is susceptible to injury which can impact its ability to act as a shock absorber. Causes of SI joint injury include trauma, degeneration, inflammation, pregnancy, ligament laxity, and muscle weakness.

The standard surgery used to address SI joint pain is sacroiliac joint fusion to completely eliminate movement at the sacroiliac joint by grafting together the ilium and sacrum. Sacroiliac fusion involves the use of implanted screws or rods, as well as a possible bone graft across the joint. Minimally-invasive procedures have been developed in recent years that improve outcomes in pain and disability, and reduce recovery time. Joint fusion can effectively reduce pain and instability caused by sacroiliac joint dysfunction or inflammation, sacroiliitis.

The present invention as described herein discloses a unique implant design that improves sacroiliac joint fusion.

### SUMMARY OF THE INVENTION

A sacroiliac fusion implant device being unitarily formed in a single piece construction using an additive manufacturing process. The implant extends along a longitudinal axis. The implant has a central core, a first latticed volume and a second latticed volume. The central core has a proximal end, a distal end and a body disposed between the proximal end and the distal end. The central core has a lumen extending along the longitudinal axis from a proximal end opening to a distal end opening configured to pass over a guide wire or pin. The distal end is tapered extending to the distal end opening. The proximal end has external threads configured for instrument engagement. The body has one or more, preferably three transverse elongated longitudinal slots or openings that extend across the lumen.

The first latticed volume is a repeating pattern formed from a cylindrical unit cell pattern. The unit cell pattern is a Gyroid Triply Periodic Minimal Surface (TPMS) pattern. The first latticed volume encompasses 60-72 percent of a total implant volume.

The second latticed volume is stochastic formed via continuous interconnected beams between seed points. The seed points are generated by a Voronoi diagram. The second latticed volume encompasses 11-13 percent of a total implant volume.

The implant has 30-50 percent porosity, preferably 40-46 percent porosity. The implant preferably has a length of 30-70 mm.

The additive manufacturing is Direct Metal Laser Sintering (DMLS) on a powder bed fusion machine, wherein the implant material is Titanium Alloy 6Al-4V ELI.

A method of implanting a sacroiliac fusion implant device has the steps of creating an insertion path for the implant; placing a guide pin through an iliac bone, across a sacroiliac joint, and into a sacrum; forming a bore using a drill over the guide pin through the iliac bone, across the sacroiliac joint, and into the sacrum; forming an insertion path along the same path as the bore, wherein the insertion path has a cross-sectional profile which differs from the cross-sectional profile of the implant to form protuberances which apply compressive forces onto the implant surfaces upon insertion to stabilize the implant within the insertion path to fuse the sacroiliac joint; and placing the implant over the guide pin and pressing the implant into the insertion path until the implant is driven into the sacrum.

### DEFINITIONS

“latticed”: Unique arrangement of connected beams forming a lightweight support structure.
“latticed volume”: A volumetric area within a device that is entirely constructed from a specific type of lattice structure.
“porosity”: Percentage of empty space or voids within the total volume of a device.
“periodic”: Type of lattice structure formed from a repeating pattern of unit cells.
“stochastic”: Type of lattice structure formed from interconnected beams between randomly generated seed points.
“unit cell”: The smallest three dimensional repeating portion of a lattice pattern.

### BRIEF DESCRIPTION OF THE DRAWINGS

The invention will be described by way of example and with reference to the accompanying drawings in which:

FIG. 1 is a distal end view of the SI fusion device of the present invention.

FIG. 2 is a cross-sectional distal end view of the SI fusion device of the present invention.

FIG. 3 is a perspective proximal end view showing the attachment screw threads of the SI fusion device of the present invention.

FIG. 4 is a perspective distal end view showing the SI fusion device of the present invention.

FIG. 5 is a plan view of the SI fusion device of the present invention.

FIG. 6 is an exemplary perspective view of the SI fusion device of the present invention.

FIGS. 7A, 7B, 7C are cross sectional views of the insertion path overlaid with the profile of the implant.

### DETAILED DESCRIPTION OF THE INVENTION

The invention is intended to stabilize the sacroiliac (SI) joint by insertion across the joint preventing rotation and motion to promote sacroiliac fusion through a minimally invasive procedure.

A typical sacroiliac fusion procedure or SI procedure typically has the following basic steps. First, the patient is laid prone face down on the operating table under general anesthesia. A small incision, usually ranging from 2 to 3 centimeters, is made in the side of the buttock and the gluteal muscles are dissected to access the space between the sacrum and the ilium into the SI joint. A small guide pin is inserted through the side of the ilium to create a small hole allowing access to the ilium. This opening is then broached or drilled through the ilium to provide passage for the implants to reach the sacrum. If a bone graft is necessary, the SI joint is cleared of cartilage and soft tissues, and the bone graft is packed into the joint space. The bone graft is typically collected from a different area of the ilium or from shavings left behind from broaching the ilium. The implant instruments are guided through the passage in the ilium, and are put into place using screws, pins, or a mallet. The incision site is then irrigated using a saline solution, which removes any debris from the wound before it is closed. Then, the incision is closed in layers using standard sutures.

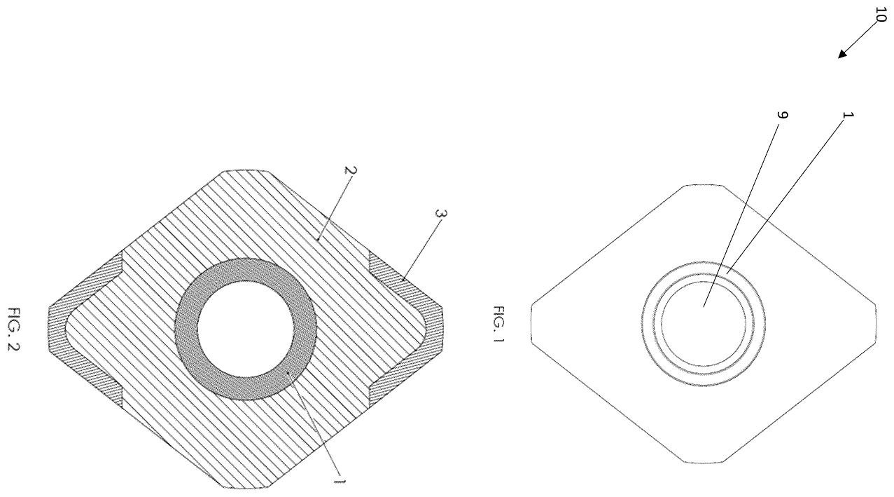

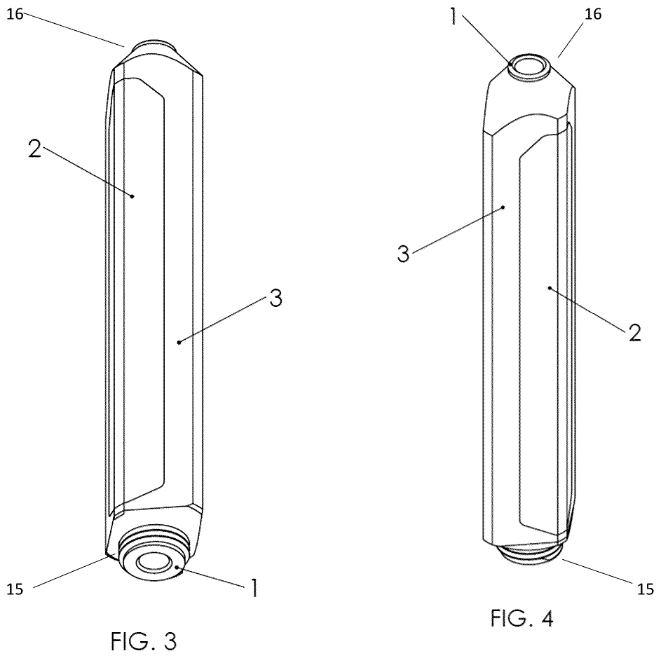

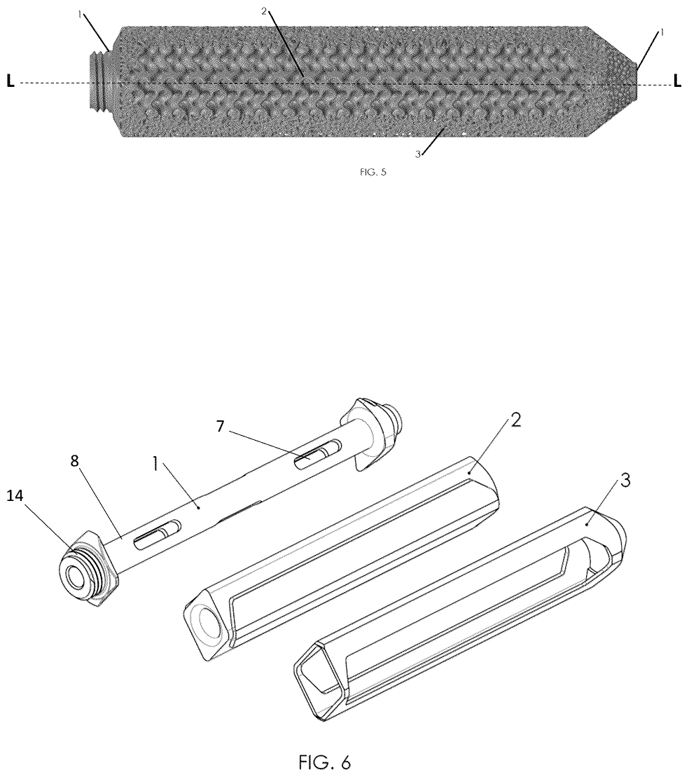

With reference to the present invention, the implant profile shown in FIG. 1 is specifically designed to minimize rotation and micromotion of the SI joint and maximize porous surface contact with bone for optimal osteointegration. Intentional regions of the implant shown in FIG. 2 are constructed of advanced custom latticing structures designed to promote fusion.

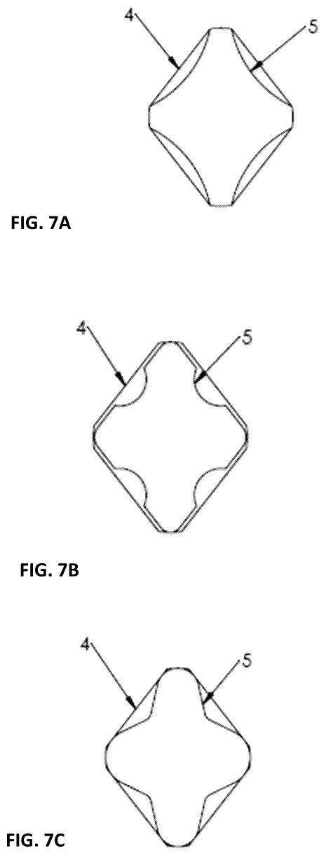

The implant 10 is made through additive manufacturing via Direct Metal Laser Sintering (DMLS) on a powder bed fusion machine from Titanium Alloy 6Al-4V ELI. The implant is available in lengths from 30-70 mm. The implant is inserted across the SI joint by an orthopedic surgeon through a minimally invasive procedure. Instrumentation is provided to create an insertion path for the implant. A guide pin is placed through an iliac bone, across the sacroiliac joint, and into a sacrum. A bore is formed using a drill over the guide pin through an iliac bone, across the sacroiliac joint, and into a sacrum. An insertion path is formed along the same path as the bore. As shown in FIGS. 7A-C, the insertion path having a cross-sectional profile 5 which differs from the cross-sectional profile 4 of the implant 10 to form protuberances which apply compressive forces onto the implant surfaces upon insertion to stabilize the implant within the insertion path to fuse the sacroiliac joint.

As shown, the implant core 1 has a distal end portion 16 with a tapered nose and proximal end portion 15 with external screw threads 14 for instrument engagement during removal or revision, and a lumen 9 along the longitudinal axis L through both ends. The core 1 is surrounded by an exterior first latticed volume 2 integrally formed with the core 1 and a second latticed volume 3 integrally formed with the core 1 and first latticed volume 2. As shown, the core 1 has a body or hollow tube body 8 with an exterior wall, the body 8 extending between the proximal 15 and distal 16 ends. The body 8 has three transverse extending slots or openings 7 extending through the body 8 across the longitudinal axis L through the exterior wall. These slots or openings 7 are elongated longitudinally.

As best shown in FIG. 2, the first latticed volume 2 is periodic and formed from a cylindrical unit cell pattern of Gyroid Triply Periodic Minimal Surface (TPMS) unit cells and encompasses 60-72% of the total implant volume. The second latticed volume 3 is stochastic and formed via continuous interconnected beams between seed points generated by the Voronoi diagram and encompasses 11-13% of the total implant volume. The resulting implant exhibits 40-46% porosity.

With reference to FIG. 6, an exemplary view of the implant 10 is shown illustrating the various portions of the implant core 1 and different lattice regions 2, 3 of the unitarily formed implant 10.

With reference to FIGS. 7A, 7B and 7C, exemplary cross-sectional views of the insertion path overlaid with the profile of the implant are illustrated.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A sacroiliac fusion implant device comprises:
a unitarily formed implant extending along a longitudinal axis, the implant having a central core, a first latticed volume and a second latticed volume; and
wherein the central core has a proximal end, a distal end and a body disposed between the proximal end and the distal end.

2. The sacroiliac fusion implant device of claim 1, wherein the unitarily formed implant is a single piece construction made by additive manufacturing.

3. The sacroiliac fusion implant device of claim 1, wherein the first latticed volume and second latticed volume are combined to a single latticed volume.

4. The sacroiliac fusion implant device of claim 1, wherein the central core has a lumen extending along the longitudinal axis from a proximal end opening to a distal end opening configured to pass over a guide wire or pin.

5. The sacroiliac fusion implant device of claim 4, wherein the distal end is tapered extending to the distal end opening.

6. The sacroiliac fusion implant device of claim 1, wherein the proximal end has external threads configured for instrument engagement.

7. The sacroiliac fusion implant device of claim 4, wherein the tube body has one or more transverse elongated longitudinal slots or openings that extend across the lumen.

8. The sacroiliac fusion implant device of claim 1, wherein the first latticed volume is a repeating pattern.

9. The sacroiliac fusion implant device of claim 8, wherein the repeating pattern is formed from a cylindrical unit cell pattern.

10. The sacroiliac fusion implant device of claim 9, wherein the unit cell pattern is a Gyroid Triply Periodic Minimal Surface (TPMS) pattern.

11. The sacroiliac fusion implant device of claim 1, wherein the first latticed volume encompasses 60-72 percent of a total implant volume.

12. The sacroiliac fusion implant device of claim 1, wherein the second latticed volume is stochastic.

13. The sacroiliac fusion implant device of claim 12, wherein the second latticed volume is formed via continuous interconnected beams between seed points.

14. The sacroiliac fusion implant device of claim 13, wherein the seed points are generated by a Voronoi diagram.

15. The sacroiliac fusion implant device of claim 1, wherein the second latticed volume encompasses 11-13 percent of a total implant volume.

16. The sacroiliac fusion implant device of claim 1, wherein the implant has 30-50 percent porosity.

17. The sacroiliac fusion implant device of claim 16, wherein the implant has 40-46 percent porosity.

18. The sacroiliac fusion implant device of claim 2, wherein the additive manufacturing is Direct Metal Laser Sintering (DMLS) on a powder bed fusion machine.

19. The sacroiliac fusion implant device of claim 1, wherein the implant has a length of 30-70 mm.

20. A method of implanting a sacroiliac fusion implant device comprises the steps of:
creating an insertion path for the implant;
placing a guide pin through an iliac bone, across a sacroiliac joint, and into a sacrum;
forming a bore using a drill over the guide pin through the iliac bone, across the sacroiliac joint, and into the sacrum;
forming an insertion path along the same path as the bore, wherein the insertion path has a cross-sectional profile which differs from the cross-sectional profile of the implant to form protuberances which apply compressive forces onto the implant surfaces upon insertion; and
placing the implant over the guide pin and passing the implant into the insertion path until the implant crosses the Sacroiliac joint.
